(19) United States
(12) Patent Application Publication
(10) Pub. No.: US 2024/0180960 A1
(43) Pub. Date: Jun. 6, 2024

(54) ADIPOSE CELL DESTRUCTION COMPONENT INCLUDING A CARBON-BASED NANOMATERIAL COMPOSITION, METHOD OF DELIVERING AN ADIPOSE CELL DESTRUCTION COMPONENT INCLUDING A CARBON-BASED NANOMATERIAL COMPOSITION, AND METHODS OF FORMING THE SAME

(71) Applicant: NABORS ENERGY TRANSITION SOLUTIONS LLC, Houston, TX (US)

(72) Inventors: Anthony G. PETRELLO, Houston, TX (US); Evan JOHNSON, Spring, TX (US); Paul YOLLIN, Tomball, TX (US); Dylan COOK, Spring, TX (US)

(21) Appl. No.: 18/529,735

(22) Filed: Dec. 5, 2023

Related U.S. Application Data
(60) Provisional application No. 63/386,214, filed on Dec. 6, 2022.

(51) Int. Cl. A61K 33/44; A61K 41/00; C01B 32/184

(52) U.S. Cl. CPC A61K 33/44; A61K 41/0052; C01B 32/184; C01B 2204/24

(57) ABSTRACT

The present disclosure relates to an adipose cell destruction method that may include preparing an adipose cell destruction component that may include a carbon-based nanomaterial composition and an adipose cell targeting composition attached to the carbon-based nanomaterial composition, delivering the adipose cell destruction component to a treatment location where the adipose cell targeting composition bonds to adipose cells, and applying a radio frequency to the adipose cell destruction component at the treatment location. The radio frequency may be configured to heat the adipose cell destruction component and destroy the adipose cells bonded to the adipose cell targeting composition at the treatment location.

### CROSS-REFERENCE TO RELATED APPLICATION(S)

This application claims priority under 35 U.S.C. § 119(c) to U.S. Provisional Patent Application No. 63/386,214, entitled “ADIPOSE CELL DESTRUCTION COMPONENT INCLUDING A CARBON-BASED NANOMATERIAL COMPOSITION, METHOD OF DELIVERING AN ADIPOSE CELL DESTRUCTION COMPONENT INCLUDING A CARBON-BASED NANOMATERIAL COMPOSITION, AND METHODS OF FORMING THE SAME,” by Anthony G. PETRELLO et al., filed on Dec. 6, 2022, which is assigned to the current assignee hereof and is incorporated herein by reference in its entirety.

### FIELD OF THE DISCLOSURE

The present disclosure relates to an adipose cell destruction component including a carbon-based nanomaterial composition, methods of delivering an adipose cell destruction component including a carbon-based nanomaterial composition, and methods of forming the same.

### SUMMARY

According to a first aspect, an adipose cell destruction method may include preparing an adipose cell destruction component that may include a carbon-based nanomaterial composition and an adipose cell targeting composition attached to the carbon-based nanomaterial composition, delivering the adipose cell destruction component to a treatment location where the adipose cell targeting composition bonds to adipose cells, and applying a radio frequency to the adipose cell destruction component at the treatment location. The radio frequency may be configured to heat the adipose cell destruction component and destroy the adipose cells bonded to the adipose cell targeting composition at the treatment location. The carbon-based nanomaterial composition may include a carbon content of at least about 60% and not greater than about 99% based on elemental analysis of the graphene composition, an oxygen content of at least about 1% and not greater than about 35% based on elemental analysis of the graphene composition, and a nitrogen content of at least about 2% and not greater than 50%.

According to another aspect, an adipose cell destruction component may include a carbon-based nanomaterial composition and an adipose cell targeting composition attached to the carbon-based nanomaterial composition. The adipose cell destruction component may be configured to be delivered to a treatment location where the adipose cell targeting composition bonds to adipose cells and heated using a radio frequency. Heating the adipose cell destruction component with the radio frequency may destroy the adipose cells bonded to the adipose cell targeting composition at the treatment location. The carbon-based nanomaterial composition may include a carbon content of at least about 60% and not greater than about 99% based on elemental analysis of the graphene composition, an oxygen content of at least about 1% and not greater than about 35% based on elemental analysis of the graphene composition, and a nitrogen content of at least about 2% and not greater than 50%.

According to still another aspect, a method of forming an adipose cell destruction component may include providing a carbon-based nanomaterial composition, attaching an adipose cell targeting composition to the carbon-based nanomaterial composition to form the adipose cell destruction component. The adipose cell destruction component may be configured to be delivered to a treatment location where the adipose cell targeting composition bonds to adipose cells and heated using a radio frequency. Heating the adipose cell destruction component with the radio frequency may destroy the adipose cells bonded to the adipose cell targeting composition at the treatment location. The carbon-based nanomaterial composition may include a carbon content of at least about 60% and not greater than about 99% based on elemental analysis of the graphene composition, an oxygen content of at least about 1% and not greater than about 35% based on elemental analysis of the graphene composition, and a nitrogen content of at least about 2% and not greater than 50%.

### BRIEF DESCRIPTION OF THE DRAWINGS

Embodiments are illustrated by way of example and are not limited to the accompanying figures.

FIG. 1 includes a diagram showing an adipose cell destruction method according to embodiments described herein;

FIG. 2 includes a diagram showing a forming method for forming an adipose cell destruction component according to embodiments described herein;

FIG. 3 includes a diagram showing a carbon-based nanomaterial composition forming method according to embodiments described herein; and

FIG. 4 includes a schematic diagram of a carbon capture system according to an embodiment of the present disclosure;

Skilled artisans appreciate that elements in the figures are illustrated for simplicity and clarity and have not necessarily been drawn to scale.

### DETAILED DESCRIPTION

The following discussion will focus on specific implementations and embodiments of the teachings. The detailed description is provided to assist in describing certain embodiments and should not be interpreted as a limitation on the scope or applicability of the disclosure or teachings. It will be appreciated that other embodiments can be used based on the disclosure and teachings as provided herein.

The terms “comprises,” “comprising,” “includes,” “including,” “has,” “having” or any other variation thereof, are intended to cover a non-exclusive inclusion. For example, a method, article, or apparatus that comprises a list of features is not necessarily limited only to those features but may include other features not expressly listed or inherent to such method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” is employed to describe elements and components described herein. This is done merely for convenience and to give a general sense of the scope of the invention. This description should be read to include one, at least one, or the singular as also including the plural, or vice versa, unless it is clear that it is meant otherwise. For example, when a single item is described herein, more than one item may be used in place of a single item. Similarly, where more than one item is described herein, a single item may be substituted for that more than one item.

Embodiments described herein are generally directed to an adipose cell destruction method using an adipose cell destruction component, an adipose cell destruction component, and a method of forming an adipose cell destruction component. According to particular embodiments, the adipose cell destruction component may include a carbon-based nanomaterial composition and an adipose cell targeting composition attached to the carbon-based nanomaterial composition. According to other embodiments, the carbon-based nanomaterial composition may be defined as any carbon-based nanomaterial composition that may include a particular carbon content and a particular oxygen content.

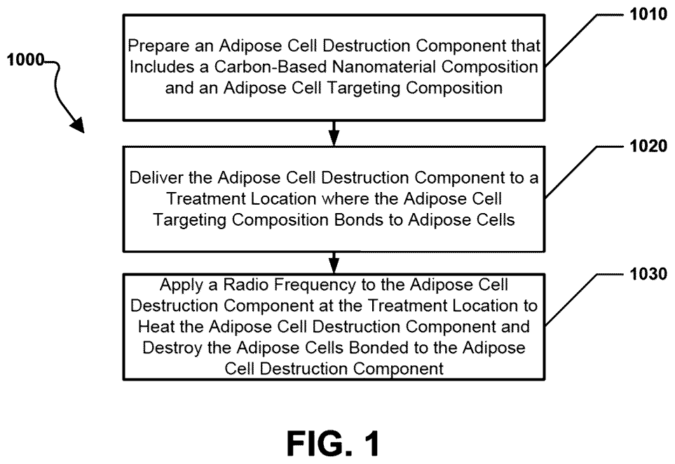

Referring first to an adipose cell destruction method using an adipose cell destruction component, FIG. 1 includes a diagram showing an adipose cell destruction method 1000 according to embodiments, described herein. According to particular embodiments, the adipose cell destruction method 1000 may include a first step 1010 of preparing an adipose cell destruction component that includes a carbon-based nanomaterial composition and an adipose cell targeting composition attached to the carbon-based nanomaterial composition, a second step 1020 of delivering the adipose cell destruction component to a treatment location where the adipose cell targeting composition bonds to adipose cells, and a third step 1030 of applying a radio frequency to the adipose cell destruction component at the treatment location.

Referring to the second step 1020, delivery of the adipose cell destruction component may be accomplished by any known delivery technology, such as injection of the adipose cell destruction component directly into the bloodstream for transport throughout the body to the treatment location or proximal injection of the adipose cell destruction component into the body at, or in the general location of, the treatment location.

According to certain embodiments, the treatment location may be any location within, or on the surface of, a body.

Referring to the third step 1030, applying the radio frequency to the adipose cell destruction component at the treatment location may include the use of any known radio frequency production apparatus, which may be used to direct radio frequency waves at the target location (i.e., direction antenna transmission of radio frequency waves) or may be used to produce radio frequency waves that fill a space encompassing the target location (i.e. non-directional antenna transmission of radio frequency waves).

According to particular embodiments, the radio frequency may be configured to heat the adipose cell destruction component and destroy the adipose cells bonded to the adipose cell destruction component at the treatment location.

According to particular embodiments, the radio frequency may include a particular frequency range. For example, the radio frequency may be at least about 100 MHz, such as, at least about 101 MHz or at least about 102 MHz or at least about 103 MHz or at least about 104 MHz or at least about 105 MHz or at least about 106 MHz or at least about 107 MHz or at least about 108 MHz or at least about 109 MHz or at least about 110 MHz or at least about 111 MHZ or at least about 112 MHz or at least about 113 MHz or at least about 114 MHz or at least about 115 MHz or at least about 116 MHz or at least about 117 MHz or at least about 118 MHz or at least about 119 MHz or even at least about 120 MHz. According to still other embodiments, the radio frequency may be not greater than about 140 MHz, such as, not greater than about 139 MHz or not greater than about 138 MHz or not greater than about 137 MHz or not greater than about 136 MHz or not greater than about 135 MHz or not greater than about 134 MHz or not greater than about 133 MHz or not greater than about 132 MHz or not greater than about 131 MHz or not greater than about 130 MHz or not greater than about 129 MHz or not greater than about 128 MHz or not greater than about 127 MHz or not greater than about 126 MHz or not greater than about 125 MHz or not greater than about 124 MHz or not greater than about 123 MHz or not greater than about 122 MHz or even not greater than about 121 MHz. It will be appreciated that the radio frequency applied to the cancer treatment delivery component may be any value between, and including, any of the minimum and maximum values noted above. It will be further appreciated that the radio frequency applied to the cancer treatment delivery component may be within a range between, and including, any of the minimum and maximum values noted above.

According to still other embodiments, the radio frequency may be transmitted at a particular power. For example, the radio frequency may be transmitted at a power of 1 watt, such as, at least about 1 watt or at least about 5 watts or at least about 10 watts or at least about 50 watts or at least about 100 watts or at least about 150 watts or at least about 200 watts or at least about 250 watts or at least about 300 watts or at least about 350 watts or at least about 400 watts or at least about 450 watts or at least about 500 watts or at least about 750 watts or at least about 1000 watts or at least about 1250 watts or at least about 1500 watts or at least about 1750 watts or even at least about 2000 watts. According to still other embodiments, the radio frequency may be not greater than about 5000 watts, such as, not greater than about 4750 watts or not greater than about 4500 watts or not greater than about 4250 watts or not greater than about 4000 watts or not greater than about 3750 watts or not greater than about 3500 watts or not greater than about 3250 watts or not greater than about 3000 watts or not greater than about 2750 watts or not greater than about 2500 watts or even not greater than about 2250 watts or not greater than about 2000 watts. It will be appreciated that the radio frequency applied to the cancer treatment delivery component may be any value between, and including, any of the minimum and maximum values noted above. It will be further appreciated that the radio frequency applied to the cancer treatment delivery component may be within a range between, and including, any of the minimum and maximum values noted above.

According to still other embodiments, application of the radio frequency may heat the treatment location to a particular temperature. For example, application of the radio frequency may heat the treatment location to a temperature of at least about 50° C., such as, at least about 55° C. or at least about 60° C. or at least about 65° C. or at least about 70° C. or at least about 75° C. or at least about 80° C. or at least about 85° C. or at least about 90° C. or at least about 95° C. or at least about 100° ° C. or at least about 105° ° C. or at least about 110° C. or at least about 115° C. or at least about 120° C. or at least about 125° C. or at least about 130° C. or at least about 135° C. or at least about 140° ° C. or at least about 145° C. or at least about 150° C. or at least about 155° C. or at least about 160° C. or at least about 165° C. or at least about 170° C. or at least about 175° C. or at least about 180° C. or at least about 185° C. or at least about 190° C. or at least about 195° C. or at least about 200° ° C. or at least about 205° C. or at least about 210° C. or at least about 215° C. or at least about 220° C. or at least about 225° C. or at least about 230° ° C. or at least about 235° C. or at least about 240° C. or at least about 245° C. or even at least about 250° ° C. According to still other embodiments, application of the radio frequency may heat the treatment location to a temperature of not greater than about 800° C., such as, not greater than about 750° C. or not greater than about 700° C. or not greater than about 650° C. or not greater than about 600° C. or not greater than about 550° C. or not greater than about 500° C. or not greater than about 450° C. or even not greater than about 400° C. It will be appreciated that application of the radio frequency may heat the treatment location to a temperature of any value between, and including, any of the minimum and maximum values noted above. It will be further appreciated that application of the radio frequency may heat the treatment location to a temperature of any value within a range between, and including, any of the minimum and maximum values noted above.

According to still other embodiments, application of the radio frequency may be applied for a particular application time length. For example, the radio frequency application time length may be at least about 0.01 seconds, such as, 0.05 seconds or at least about 0.1 seconds or at least about 0.2 seconds or at least about 0.3 seconds or at least about 0.4 seconds or at least about 0.5 seconds or at least about 0.6 seconds or at least about 0.7 seconds or at least about 0.8 seconds or at least about 0.9 seconds or at least about 1.0 seconds or at least about 5.0 seconds or at least about 10 seconds or at least about 15 seconds or at least about 20 seconds or at least about 25 seconds or even at least about 30 seconds. According to still other embodiments, the radio frequency application time length may be not greater than about 60 seconds, such as, not greater than about 55 seconds or not greater than about 50 seconds or not greater than about 45 seconds or not greater than about 40 seconds or even not greater than about 35 seconds. It will be appreciated that the radio frequency application time length may be any value between, and including, any of the minimum and maximum values noted above. It will be further appreciated that the radio frequency application time length may be any value within a range between, and including, any of the minimum and maximum values noted above.

According to still other embodiments, the radio frequency may be applied in a sequence of bursts, each burst being applied for a particular burst application time length. For example, the radio frequency bust application time length may be at least about 0.01 seconds, such as, 0.05 seconds or at least about 0.1 seconds or at least about 0.2 seconds or at least about 0.3 seconds or at least about 0.4 seconds or at least about 0.5 seconds or at least about 0.6 seconds or at least about 0.7 seconds or at least about 0.8 seconds or at least about 0.9 seconds or at least about 1.0 seconds or at least about 5.0 seconds or at least about 10 seconds or at least about 15 seconds or at least about 20 seconds or at least about 25 seconds or even at least about 30 seconds. According to still other embodiments, the radio frequency burst application time length may be not greater than about 60 seconds, such as, not greater than about 55 seconds or not greater than about 50 seconds or not greater than about 45 seconds or not greater than about 40 seconds or even not greater than about 35 seconds. It will be appreciated that the radio frequency burst application time length may be any value between, and including, any of the minimum and maximum values noted above. It will be further appreciated that the radio frequency burst application time length may be any value within a range between, and including, any of the minimum and maximum values noted above. It will be appreciated that the sequence of busts may be applied for a particular application time length equal to the application time length described herein.

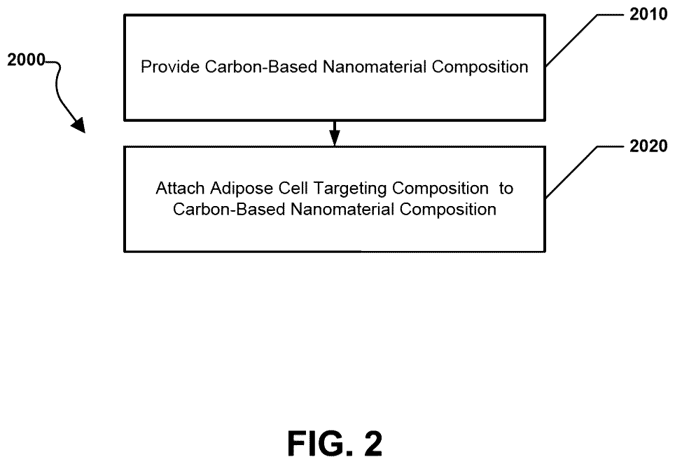

Referring now to an adipose cell destruction component formed according to embodiments described herein. FIG. 2 includes a diagram showing a forming method 2000 for forming an adipose cell destruction component according to embodiments described herein. According to particular embodiments, the forming method 2000 may include a first step 2010 providing a carbon-based nanomaterial composition, and a second step 2020 of attaching an adipose cell targeting composition to the carbon-based nanomaterial composition.

Referring to the second step 2020, attaching the adipose cell targeting composition to the carbon-based nanomaterial composition may be through a functional bond between the adipose cell targeting composition and the carbon-based nanomaterial composition or through absorption of the adipose cell targeting composition into the carbon-based nanomaterial. According to still other embodiments, the adipose cell targeting composition may be absorbed within layers of the carbon-based nanomaterial. For example, if the carbon-based nanomaterial has a sheet structure, the adipose cell targeting composition may be absorbed within and bond to layers of the sheet structure. According to other embodiments, if the carbon-based nanomaterial has a nano-onion structure, i.e., a structure that includes layers folded over on themselves such that they resemble an onion shell, the adipose cell targeting composition may be absorbed within and bond to layers of the nano-onion structure.

According to yet other embodiments, the adipose cell targeting composition may include any molecule that is specifically attracted to an adipose cell.

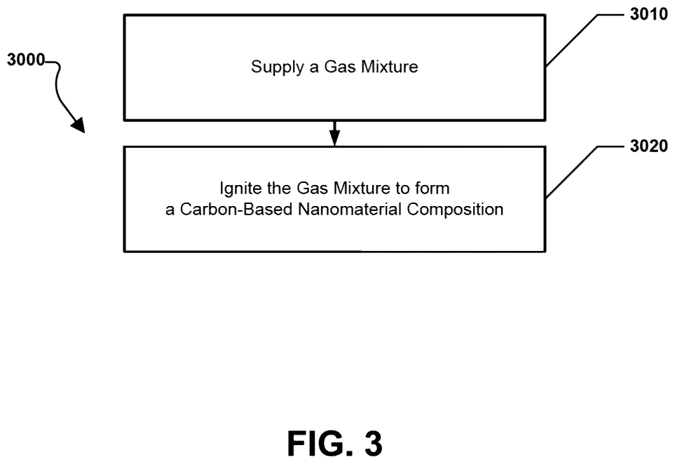

Referring to first step 2010 of providing a carbon-based nanomaterial composition, FIG. 3 includes a diagram showing a forming method 3000 for forming a carbon-based nanomaterial composition according to embodiments described herein. According to particular embodiments, the forming method 3000 may include a first step 3010 of supplying a gas mixture, and a second step 3020 of igniting the gas mixture to form the carbon-based nanomaterial composition.

Referring to first step 3010, according to particular embodiments, the gas mixture may include acetylene gas, and oxygen gas. According to still other embodiments, the gas mixture may further include hydrogen gas.

According to a certain embodiment, the gas mixture may include a particular molar ratio AGₘₒₗ/GMₘₒₗ, where the AGₘₒₗ is equal to the moles of acetylene gas in the gas mixture and GMₘₒₗ is equal to the total moles of gas in the gas mixture. For example, the gas mixture may include a molar ratio AGₘₒₗ/GMₘₒₗ of at least about 0.20, such as, at least about 0.21 or at least about 0.22 or at least about 0.23 or at least about 0.24 or at least about 0.25 or at least about 0.26 or at least about 0.27 or at least about 0.28 or at least about 0.29 or at least about 0.30 or at least about 0.31 or at least about 0.32 or at least about 0.33 or at least about 0.34 or even at least about 0.35. According to still other embodiments, the gas mixture may include a molar ratio AGₘₒₗ/GMₘₒₗ of not greater than about 0.99, such as, not greater than about 0.95 or not greater than about 0.90 or not greater than about 0.85 or not greater than about 0.80 or not greater than about 0.75 or not greater than about 0.70 or not greater than about 0.65 or even not greater than about 0.60. It will be appreciated that the gas mixture may include a molar ratio AGₘₒₗ/GMₘₒₗ of any value between, and including, any of the minimum and maximum values noted above. It will be further appreciated that the gas mixture may include a molar ratio AGₘₒₗ/GMₘₒₗ within a range between, and including, any of the minimum and maximum values noted above.

According to a certain embodiment, the gas mixture may include a particular molar ratio OGₘₒₗ/GMₘₒₗ, where the OGₘₒₗ is equal to the moles of oxygen gas in the gas mixture and GMₘₒₗ is equal to the total moles of gas in the gas mixture. For example, the gas mixture may include a molar ratio OGₘₒₗ/GMₘₒₗ of at least about 0.01, such as, at least about 0.02 or at least about 0.03 or at least about 0.04 or at least about 0.05 or at least about 0.06 or at least about 0.07 or at least about 0.08 or at least about 0.09 or at least about 0.10 or even at least about 0.11 or at least about 0.12 or at least about 0.13 or at least about 0.14 or at least about 0.15 or at least about 0.16 or at least about 0.17 or at least about 0.18 or at least about 0.19 or at least about 0.20 or at least about 0.25 or at least about 0.30 or at least about 0.35 or even at least about 0.40. According to still other embodiments, the gas mixture may include a molar ratio OGₘₒₗ/GMₘₒₗ of not greater than about 0.85, such as, not greater than about 0.80 or not greater than about 0.75 or not greater than about 0.70 or not greater than about 0.65 or not greater than about 0.60 or not greater than about 0.55 or not greater than about 0.54 or not greater than about 0.53 or not greater than about 0.52 or not greater than about 0.51 or even not greater than about 0.50. It will be appreciated that the gas mixture may include a molar ratio OGₘₒₗ/GMₘₒₗ of any value between, and including, any of the minimum and maximum values noted above. It will be further appreciated that the gas mixture may include a molar ratio OGₘₒₗ/GMₘₒₗ within a range between, and including, any of the minimum and maximum values noted above.

According to a certain embodiment, the gas mixture may include a particular molar ratio HGₘₒₗ/GMₘₒₗ, where the HGₘₒₗ is equal to the moles of hydrogen gas in the gas mixture and GMₘₒₗ is equal to the total moles of gas in the gas mixture. For example, the gas mixture may include a molar ratio HGₘₒₗ/GMₘₒₗ of at least about 0.05, such as, at least about 0.10 or at least about 0.15 or at least about 0.20 or at least about 0.25 or at least about 0.30 or even at least about 0.35. According to still other embodiments, the gas mixture may include a molar ratio HGₘₒₗ/GMₘₒₗ of not greater than about 0.99, such as, not greater than about 0.95 or not greater than about 0.90 or not greater than about 0.85 or not greater than about 0.80 or not greater than about 0.75 or not greater than about 0.70 or not greater than about 0.65 or not greater than about 0.60 or not greater than about 0.55 or not greater than about 0.50 or not greater than about 0.45 or even not greater than about 0.40. It will be appreciated that the gas mixture may include a molar ratio HGₘₒₗ/GMₘₒₗ of any value between, and including, any of the minimum and maximum values noted above. It will be further appreciated that the gas mixture may include a molar ratio HGₘₒₗ/GMₘₒₗ within a range between, and including, any of the minimum and maximum values noted above.

According to a certain embodiment, the gas mixture may further include methane gas. According to still other embodiments, the gas mixture may include a particular molar ratio MGₘₒₗ/GMₘₒₗ, where the MGₘₒₗ is equal to the moles of methane gas in the gas mixture and GMₘₒₗ is equal to the total moles of gas in the gas mixture. For example, the gas mixture may include a molar ratio MGₘₒₗ/GMₘₒₗ of at least about 0.25, such as, at least about 0.26 or at least about 0.27 or at least about 0.28 or at least about 0.29 or at least about 0.30 or at least about 0.31 or at least about 0.32 or at least about 0.33 or at least about 0.34 or even at least about 0.35. According to still other embodiments, the gas mixture may include a molar ratio MGₘₒₗ/GMₘₒₗ of not greater than about 0.99, such as, not greater than about 0.95 or not greater than about 0.90 or not greater than about 0.85 or not greater than about 0.80 or not greater than about 0.75 or not greater than about 0.70 or not greater than about 0.65 or even not greater than about 0.60. It will be appreciated that the gas mixture may include a molar ratio MGₘₒₗ/GMₘₒₗ of any value between, and including, any of the minimum and maximum values noted above. It will be further appreciated that the gas mixture may include a molar ratio MGₘₒₗ/GMₘₒₗ within a range between, and including, any of the minimum and maximum values noted above.

According to particular embodiments, the gas mixture may include a particular content of acetylene gas. For example, the gas mixture may include acetylene gas at a concentration of at least about 1.2 mol, such as, at least about 1.4 mol or at least about 1.6 mol or at least about 1.8 mol or at least about 2.0 mol or at least about 2.05 mol or at least about 2.06 mol or at least about 2.07 mol or at least about 2.08 mol or at least about 2.09 mol or at least about 2.10 mol or at least about 2.11 mol or at least about 2.12 mol or at least about 2.13 mol or at least about 2.14 mol or at least about 2.15 mol or at least about 2.16 mol or at least about 2.17 mol or at least about 2.18 mol or at least about 2.19 mol or at least about 2.20 mol or at least about 2.25 mol or at least about 2.30 mol or at least about 2.35 mol or at least about 2.40 mol or at least about 2.45 mol or at least about 2.50 mol or at least about 2.75 mol or at least about 3.0 mol or at least about 3.5 mol or at least about 4.0 mol or at least about 4.5 mol or at least about 5.0 mol or at least about 5.5 mol or at least about 6.0 mol or even at least about 6.5 mol. According to still other embodiments, the gas mixture may include acetylene gas at a concentration of not greater than about 18.0 mol, such as, not greater than about 17.0 mol or not greater than about 16.0 mol or not greater than about 15.0 mol or not greater than about 14.0 mol or not greater than about 13.0 mol or not greater than about 12.0 mol or not greater than about 11.0 mol or not greater than about 10.0 mol or not greater than about 9.0 mol or even not greater than about 8.0 mol. It will be appreciated that the acetylene gas concentration in the gas mixture may be any value between, and including, any of the minimum and maximum values noted above. It will be further appreciated that the acetylene gas concentration in the gas mixture may be within a range between, and including, any of the minimum and maximum values noted above.

According to other embodiments, the gas mixture may include a particular content of oxygen gas. For example, the gas mixture may include oxygen gas at a concentration of at least about 0.3 mol, such as, at least about 0.31 mol or at least about 0.32 mol or at least about 0.33 mol or at least about 0.34 mol or at least about 0.35 mol or at least about 0.36 mol or at least about 0.37 mol or at least about 0.38 mol or at least about 0.39 mol or at least about 0.40 mol or at least about 0.41 mol or at least about 0.42 mol or at least about 0.43 mol or at least about 0.44 mol or at least about 0.45 mol or at least about 0.46 mol or at least about 0.47 mol or at least about 0.48 mol or even at least about 0.49 mol. According to still other embodiments, the gas mixture may include oxygen gas at a concentration of not greater than about 12 mol, such as, not greater than about 10 mol or not greater than about 8.0 mol or not greater than about 6.0 mol or not greater than about 4.0 mol or not greater than about 2.0 mol or not greater than about 1.0 mol or not greater than about 0.75 mol or even not greater than about 0.5 mol. It will be appreciated that the oxygen gas concentration in the gas mixture may be any value between, and including, any of the minimum and maximum values noted above. It will be further appreciated that the oxygen gas concentration in the gas mixture may be within a range between, and including, any of the minimum and maximum values noted above.

According to still other embodiments, the gas mixture may include a particular content of hydrogen gas. For example, the gas mixture may include hydrogen gas at a concentration of at least about 0.60 mol, such as, at least about 0.61 mol or at least about 0.62 mol or at least about 0.63 mol or at least about 0.64 mol or at least about 0.65 mol or at least about 0.66 mol or at least about 0.67 mol or at least about 0.68 mol or at least about 0.69 mol or at least about 0.70 mol or at least about 0.71 mol or at least about 0.72 mol or at least about 0.73 mol or at least about 0.74 mol or at least about 0.75 mol or at least about 0.76 mol or at least about 0.77 mol or at least about 0.78 mol or at least about 0.79 mol or even at least about 0.80 mol. According to still other embodiments, the gas mixture may include hydrogen gas at a concentration of not greater than about 20.0 mol, such as, not greater than about 15 mol or not greater than about 10.0 mol or not greater than about 5.0 mol or not greater than about 4.0 mol or not greater than about 3.5 mol or not greater than about 3.0 mol or not greater than about 2.5 mol or not greater than about 2.0 mol or not greater than about 1.5 mol or even not greater than about 1.0 mol. It will be appreciated that the hydrogen gas concentration in the gas mixture may be any value between, and including, any of the minimum and maximum values noted above. It will be further appreciated that the hydrogen gas concentration in the gas mixture may be within a range between, and including, any of the minimum and maximum values noted above.

According to particular embodiments, the gas mixture may include a particular content of methane gas. For example, the gas mixture may include methane gas at a concentration of at least about 1.2 mol, such as, at least about 1.4 mol or at least about 1.6 mol or at least about 1.8 mol or at least about 2.0 mol or at least about 2.05 mol or at least about 2.06 mol or at least about 2.07 mol or at least about 2.08 mol or at least about 2.09 mol or at least about 2.10 mol or at least about 2.11 mol or at least about 2.12 mol or at least about 2.13 mol or at least about 2.14 mol or at least about 2.15 mol or at least about 2.16 mol or at least about 2.17 mol or at least about 2.18 mol or at least about 2.19 mol or at least about 2.20 mol or at least about 2.25 mol or at least about 2.30 mol or at least about 2.35 mol or at least about 2.40 mol or at least about 2.45 mol or at least about 2.50 mol or at least about 2.75 mol or at least about 3.0 mol or at least about 3.5 mol or at least about 4.0 mol or at least about 4.5 mol or at least about 5.0 mol or at least about 5.5 mol or at least about 6.0 mol or even at least about 6.5 mol. According to still other embodiments, the gas mixture may include methane gas at a concentration of not greater than about 18.0 mol, such as, not greater than about 17.0 mol or not greater than about 16.0 mol or not greater than about 15.0 mol or not greater than about 14.0 mol or not greater than about 13.0 mol or not greater than about 12.0 mol or not greater than about 11.0 mol or not greater than about 10.0 mol or not greater than about 9.0 mol or even not greater than about 8.0 mol. It will be appreciated that the methane gas concentration in the gas mixture may be any value between, and including, any of the minimum and maximum values noted above. It will be further appreciated that the methane gas concentration in the gas mixture may be within a range between, and including, any of the minimum and maximum values noted above.

Referring now to embodiments of the carbon-based nanomaterial composition formed according to forming method 100, the carbon-based nanomaterial composition may include particular carbon content based on elemental analysis conducted using x-ray photoelectron spectroscopy (XPS). For example, the carbon-based nanomaterial composition may include a carbon content of at least about 75.0%, such as, at least about 78.0% or at least about 80.0% or at least about 83% or at least about 85% or at least about 88% or at least about 90% or at least about 91% or at least about 92% or at least about 93% or at least about 94.0% or even at least about 95.0%. According to still other embodiments, the carbon-based nanomaterial composition may include a carbon content of not greater than about 100%, such as, not greater than about 99.5% or not greater than about 99% or not greater than about 98.5% or not greater than about 98% or not greater than about 97.5% or not greater than about 97% or not greater than about 96.5% or even not greater than about 96.0%. It will be appreciated that the carbon content in the carbon-based nanomaterial composition may be any value between, and including, any of the minimum and maximum values noted above. It will be further appreciated that the carbon content in the carbon-based nanomaterial composition may be within a range between, and including, any of the minimum and maximum values noted above.

According to still other embodiments, the carbon-based nanomaterial composition may include particular oxygen content based on elemental analysis conducted using x-ray photoelectron spectroscopy (XPS). For example, the carbon-based nanomaterial composition may include an oxygen content of at least about 0.0%, such as, at least about 0.5% or at least about 1.0% or at least about 1.5% or at least about 2.0% or at least about 2.5% or at least about 3.0% or at least about 3.5% or at least about 4.0% or at least about 4.5% or even at least about 5.0%. According to still other embodiments, the carbon-based nanomaterial composition may include an oxygen content of not greater than about 25%, such as, not greater than about 23% or not greater than about 20% or not greater than about 18% or not greater than about 15% or not greater than about 13% or not greater than about 10% or not greater than about 8% or even not greater than about 6.0%. It will be appreciated that the oxygen content in the carbon-based nanomaterial composition may be any value between, and including, any of the minimum and maximum values noted above. It will be further appreciated that the oxygen content in the carbon-based nanomaterial composition may be within a range between, and including, any of the minimum and maximum values noted above.

According to still other embodiments, the carbon-based nanomaterial composition may have a particular D/G ratio as measured by performing x-ray photoelectron spectroscopy on a sample of powder and detangling the spectrum produced. For example, the carbon-based nanomaterial composition may have a D/G ratio of at least about 0.1, such as, at least about 0.15 or at least about 0.20 or at least about 0.25 or at least about 0.30 or at least about 0.35 or at least about 0.40 or at least about 0.45. According to still other embodiments, the carbon-based nanomaterial composition may have a D/G ratio of not greater than about 2.0, such as, not greater than about 1.95 or not greater than about 1.90 or not greater than about 1.85 or not greater than about 1.80 or not greater than about 1.75 or not greater than about 1.70 or not greater than about 1.65 or not greater than about 1.60 or not greater than about 1.55 or not greater than about 1.50 or not greater than about 1.45 or not greater than about 1.40 or not greater than about 1.35 or not greater than about 1.30 or not greater than about 1.25 or not greater than about 1.20 or not greater than about 1.15 or not greater than about 1.10 or not greater than about 1.05 or not greater than about 1.00 or not greater than about 0.95 or not greater than about 0.9 or not greater than about 0.85 or not greater than about 0.8 or not greater than about 0.75 or not greater than about 0.7 or not greater than about 0.65 or even not greater than about 0.6. It will be appreciated that the D/G ratio of the carbon-based nanomaterial composition may be any value between, and including, any of the minimum and maximum values noted above. It will be further appreciated that the D/G ratio of the carbon-based nanomaterial composition may be within a range between, and including, any of the minimum and maximum values noted above.

According to still other embodiments, the carbon-based nanomaterial composition may have a particular aspect ratio as measured by dividing the lateral size by the thickness of a given sample. For example, the carbon-based nanomaterial composition may have an aspect ratio of at least about 1.0, such as, at least about 5 or at least about 10 or at least about 15. According to still other embodiments, the carbon-based nanomaterial composition may have an aspect ratio of not greater than about 100, such as, not greater than about 95 or not greater than about 90 or not greater than about 85 or not greater than about 80 or not greater than about 75 or not greater than about 70 or not greater than about 65 or even not greater than about 60. It will be appreciated that the aspect ratio of the carbon-based nanomaterial composition may be any value between, and including, any of the minimum and maximum values noted above. It will be further appreciated that the aspect ratio of the carbon-based nanomaterial composition may be within a range between, and including, any of the minimum and maximum values noted above.

According to yet other embodiments, the carbon-based nanomaterial composition may have a particular carbon hybridization ratio Pₛₚ₃/Pₛₚ₂, where Pₛₚ₃ is the percent of carbon within the carbon-based nanomaterial composition having a sp3 hybridization and Pₛₚ₂ is the percent of carbon within the carbon-based nanomaterial composition having a sp2 hybridization. For example, the carbon-based nanomaterial composition may have a carbon hybridization ratio Pₛₚ₃/Pₛₚ₂ of at least about 0.0, such as, at least about 0.1 or at least about 0.2 or at least about 0.3 or at least about 0.4 or at least about 0.5 or at least about 0.6 or at least about 0.7 or at least about 0.8 or at least about 0.9 or at least about 1.0 or at least about 1.1 or at least about 1.2 or at least about 1.3 or at least about 1.4 or even at least about 1.5. According to still other embodiments, the carbon-based nanomaterial composition may have a carbon hybridization ratio Pₛₚ₃/Pₛₚ₂ of not greater than about 5.00, such as, not greater than about 4.75 or not greater than about 4.5 or not greater than about 4.25 or not greater than about 4.0 or not greater than about 3.75 or not greater than about 3.50 or not greater than about 3.25 or not greater than about 3.0 or not greater than about 2.9 or not greater than about 2.8 or not greater than about 2.7 or not greater than about 2.6 or not greater than about 2.5 or not greater than about 2.4 or not greater than about 2.3 or not greater than about 2.2 or not greater than about 2.1 or even not greater than about 2.0. It will be appreciated that the carbon hybridization ratio Pₛₚ₃/Pₛₚ₂ of the carbon-based nanomaterial composition may be any value between, and including, any of the minimum and maximum values noted above. It will be further appreciated that the carbon hybridization ratio Pₛₚ₃/Pₛₚ₂ of the carbon-based nanomaterial composition may be within a range between, and including, any of the minimum and maximum values noted above.

According to certain embodiments, the carbon-based nanomaterial composition may have particular carbon structures. For example, according to certain embodiments, the carbon-based nanomaterial composition may include carbon-based nanosheets. According to certain embodiments, the carbon-based nanomaterial composition may consist of carbon-based nanosheets. For purposes of embodiments described herein, a nanosheet may be defined as a two-dimensional allotropic form of carbon. According to still other embodiments, a nanosheet may have Sp2-hybridized carbon atoms, connected by sigma and pi bonds in a hexagonal lattice of polyaromatic rings.

According to certain embodiments, the carbon-based nanomaterial composition may include carbon-based nanoflakes. According to certain embodiments, the carbon-based nanomaterial composition may consist of carbon-based nanoflakes. For purposes of embodiments described herein, a nanoflake may be defined as a Lamellae of graphene, such as, a two-dimensional carbon sheet. According to still other embodiments, the nanoflakes may have a two-dimensional carbon sheet size of between about 50 nm and 100 nm.

According to certain embodiments, the carbon-based nanomaterial composition may include carbon-based nanospheres. According to certain embodiments, the carbon-based nanomaterial composition may consist of carbon-based nanospheres. For purposes of embodiments described herein, a nanosphere may be defined as a Sp2-hybridized form of carbon with atomic carbon clusters formed into a spherical structure via covalent bonds. According to certain embodiments, the nanospheres can have radii ranging from about 50 nm to about 250 nm.

According to certain embodiments, the carbon-based nanomaterial composition may include carbon-based nano-onions. According to certain embodiments, the carbon-based nanomaterial composition may consist of carbon-based nano-onions. For purposes of embodiments described herein, a nano-onion may be defined as a nanostructure that includes multiple concentric shells of hexagonal-latticed sheets, strained to form spherical structures. According to still other embodiments, the nano-onions may include layers folded over on themselves such that they resemble an onion shell, sometimes encompassing a small volume of amorphous carbon.

According to still other embodiments, the carbon-based nanomaterial composition may include carbon black. According to certain embodiments, the carbon-based nanomaterial composition may consist of carbon black. For purposes of embodiments described herein, carbon black may be defined as material that is spherical with radii below 1000 nm. According to still other embodiments, the carbon black may be amorphous and may be a black fine powder.

According to still other embodiments, the carbon-based nanomaterial composition may include turbostratic carbon. According to certain embodiments, the carbon-based nanomaterial composition may consist of turbostratic carbon. For purposes of embodiments described herein, turbostratic carbon may be defined as a material having a mixture of sp2- and sp3-hybridized carbon, where the sp2-hybridized planes are surrounded and connected by a sp3-hybridized amorphous matrix. The turbostratic carbon may include curved sheets of graphene-like carbon-polyaromatic structures, forming grape-like fractal aggregates of primary particles.

According to still other embodiments, the carbon-based nanomaterial composition may include any combination of carbon-based nanosheets, carbon-based nanoflakes, carbon-based nanospheres, carbon-based nano-onions, carbon black, or turbostratic carbon. According to still other embodiments, the carbon-based nanomaterial composition may consist of any combination of carbon-based nanosheets, carbon-based nanoflakes, carbon-based nanospheres, carbon-based nano-onions, carbon black, or turbostratic carbon.

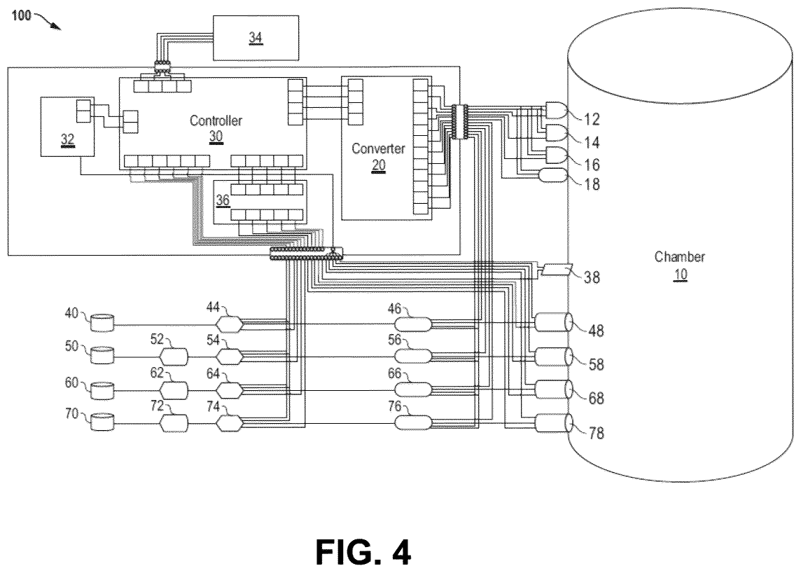

Turning now to a system for synthesis of carbon-based nanomaterial composition according to embodiments described herein, FIG. 4 includes a diagram of a carbon capture system according to embodiments described herein. As shown in FIG. 4, a carbon capture system 100 according to embodiments of the present disclosure includes a combustion chamber 10 for conversion of hydrocarbon gas or liquid into carbon-based nanomaterial composition. The system 100 may be scaled as needed and may be located onsite, for example, at a hydrocarbon drilling operation or other suitable hydrocarbon feedstock site. Advantageously, the apparatus and methods disclosed herein permit a wide range of hydrocarbons to be used as a feedstock thereby converting numerous types of carbon-containing fluids, such as industrial flue gas output, to generate a valuable product, e.g., carbon-based nanomaterial composition. Thus, the disclosure herein beneficially teaches to capture a variety of carbon in industrial outputs and minimize greenhouse gas emissions therefrom while providing a valuable product for further industrial processes, materials, and equipment, for example, carbon-based nanomaterial composition-coated proton electron membranes. The combustion chamber 10 of FIG. 4 may be a heavy-duty chamber with multiple injection ports for controlled injection of the hydrocarbon material and separate injection of oxygen and hydrogen that forces re-bonding of carbon, hydrogen, and oxygen when ignited to form carbon-based nanomaterial composition and other products that do not contribute to greenhouse gas emissions, such as water. Without being bound by theory, the use of controlled, separate injection of oxygen and hydrogen allows for a much faster combustion of the hydrocarbon material as compared with traditional oxidizing agents; this permits a more complete breakdown of the hydrocarbon material. The combustion chamber 10 may be formed of any suitable material, such as aluminum, titanium aluminum, nickel aluminum, cast iron, steel, and the like. In some embodiments, the combustion chamber 10 is configured to withstand at least 1000 psi of internal pressure.

The combustion chamber 10 may include one or more sensors configured to monitor and measure conditions within the combustion chamber 10. In some embodiments, the combustion chamber 10 includes a temperature sensor 18 configured to measure a temperature within the combustion chamber 10. In some embodiments, the combustion chamber 10 includes a low pressure sensor 16, a pressure sensor 14, and a high pressure sensor 12, each configured to measure a pressure within the combustion chamber 10. In one or more embodiments, the combustion chamber 10 may include an opacity sensor configured to measure an opacity within the combustion chamber 10. In some embodiments, the combustion chamber 10 may include a vacuum valve configured to create a vacuum within the combustion chamber 10 as a precursor to introducing any reactants (or inert gas). In some embodiments, the combustion chamber 10 includes a pressure release valve configured to release pressure from the combustion chamber 10. The pressure release valve may be actuated once a threshold pressure is reached within the combustion chamber 10 and/or on demand, for example, at a set time after each combustion within the combustion chamber 10.

The system includes an inert gas source 40, a flue gas source 50, an oxygen source 60, and a hydrogen source 70 each in fluidic communication with the combustion chamber 10. The inert gas source 40 is arranged to provide a supply of an inert gas, such as argon, under pressure to the combustion chamber 10, wherein said pressure may be monitored by a pressure sensor 44. The inert gas provides an inert environment for clean combustion within the combustion chamber 10. For instance, the inert environment may prevent or suppress formation of NOx (nitrogen oxides) that might otherwise occur. A flow meter 46 is provided between the inert gas source 40 and the combustion chamber 10 and the flow meter 46 is configured to measure a flow rate of inert gas from the inert gas source 40 into the combustion chamber 10. The inert gas is introduced into the combustion chamber 10 through an injection port 48, which may include a one-way valve in order to maintain pressure within the combustion chamber 10 and avoid flashback. In some embodiments, the one-way valve is a solenoid valve.

The flue gas source 50 supplies a carbon-based gas or liquid to the combustion chamber 10. Suitable carbon-based gases or liquids include a variety of commercial and industrial output products that include carbon, typically in a hydrocarbon, which include but are not limited to carbon dioxide, methane, propane, acetylene, butane, or combinations thereof. The carbon content of the carbon-based gases or liquids is not particularly limited. In some embodiments, the flue gas source 50 is an exhaust stream from an industrial reaction process, such as a coal energy plant, a drilling operation, a combustion engine, or a landfill. In other embodiments, the exhaust stream from said industrial reaction process may be collected and stored in a tank or other vessel that may be used later in the system 100. In some embodiments, the flue gas source 50 comprises a holding tank configured to receive and pressurize the exhaust stream from such an industrial process to provide a consistent feedstock pressure to the apparatus herein. In any embodiment, the flue gas source 50 may include a pressure sensor 54 in communication therewith configured to monitor a pressure of the carbon-based gas or liquid from the flue gas source 50. Between the flue gas source 50 and the combustion chamber 10 is a flow meter 56 configured to measure a flow rate of the carbon-based gas or liquid from the flue gas source 50 into the combustion chamber 10. The carbon-based gas or liquid is introduced into the combustion chamber 10 through an injection port 58, which may include a one-way valve in order to maintain pressure within the combustion chamber 10 and avoid flashback. In some embodiments, the one-way valve is a solenoid valve. In some embodiments, a flash arrester 52 may also be included between the flue gas source 50 and the combustion chamber 10, e.g., between the pressure sensor 54 and the flue gas source 50. The flash arrester 52 may include a sensor configured to detect flashback during the combustion process in the combustion chamber 10 and, in response, shut down the system 100 to minimize or avoid the risk of explosion or fire.

The oxygen source 60 supplies oxygen gas to the combustion chamber 10. In some embodiments, the oxygen source 60 is pressurized at about 50 psi or greater. In some embodiments, the oxygen source 60 receives oxygen from a proton exchange membrane (PEM) electrolyzer and, optionally, pressurizes the oxygen. In other embodiments, the oxygen source 60 comprises an oxygen cylinder. In any embodiment, the oxygen source 60 may include a pressure sensor 64 in communication therewith configured to monitor a pressure of the oxygen from the oxygen source 60. Between the oxygen source 60 and the combustion chamber 10 is a flow meter 66 configured to measure a flow rate of the oxygen from the oxygen source 60 into the combustion chamber 10. The oxygen is introduced into the combustion chamber 10 through an injection port 68, which may include a one-way valve in order to maintain pressure within the combustion chamber 10 and avoid flashback. In some embodiments, the one-way valve is a solenoid valve. In some embodiments, a flash arrester 62 may also be included between the oxygen source 60 and the combustion chamber 10, e.g., between the pressure sensor 64 and the oxygen source 60. The flash arrester 62 may include a sensor configured to detect flashback during the combustion process in the combustion chamber 10 and, in response, shut down the system 100.

The hydrogen source 70 supplies hydrogen gas to the combustion chamber 10. In some embodiments, the hydrogen source 70 is pressurized at about 50 psi or greater. In some embodiments, the hydrogen source 70 receives hydrogen from a proton exchange membrane (PEM) electrolyzer and, optionally, pressurizes the hydrogen. In other embodiments, the hydrogen source 70 comprises a hydrogen cylinder. In any embodiment, the hydrogen source 70 may include a pressure sensor 74 in communication therewith configured to monitor a pressure of the hydrogen from the hydrogen source 70. Between the hydrogen source 70 and the combustion chamber 10 is a flow meter 76 configured to measure a flow rate of the hydrogen from the hydrogen source 70 into the combustion chamber 10. The hydrogen is introduced into the combustion chamber 10 through an injection port 78, which may include a one-way valve in order to maintain pressure within the combustion chamber 10 and avoid flashback. In some embodiments, the one-way valve is a solenoid valve. In some embodiments, a flash arrester 72 may also be included between the hydrogen source 70 and the combustion chamber 10, e.g., between the pressure sensor 74 and the hydrogen source 70. The flash arrester 72 may include a sensor configured to detect flashback during the combustion process in the combustion chamber 10 and, in response, shut down the system 100.

The combustion chamber 10 includes an ignition device 38, such as a spark plug. The ignition device 38 is configured to initiate a series of precisely timed combustions. For example, each combustion event may last about a millisecond. The spacing between combustions and the duration of combustions may be appropriately adjusted based on the measured conditions of the system 100. In one or more embodiments, the ignition device 38 is positioned at a mid-point of the combustion chamber 10. According to this configuration, as particles of the reactants (flue gas, oxygen, and hydrogen) accelerate in each direction the particles hit at each end and assemble the carbon-based nanomaterial composition.

The system 100 also includes a controller 30 configured to receive inputs from the sensors within the system 100 and to control combustion conditions within the combustion chamber 10. In some embodiments, the controller 30 in configured to receive inputs from one or more of the flow meters 46, 56, 66, 76, the temperature sensor 18, the low pressure sensor 16, the pressure sensor 14, the high pressure sensor 12, and the pressure sensors 44, 54, 64, 74. In some embodiments, the controller 30 comprises a converter 20 configured to receive said inputs as analog signals and convert the analog signals into digital signals.

The controller 30 may also include a driver 36. In some embodiments, the driver 36 is configured to actuate one or more of the solenoid valves at injection ports 48, 58, 68, 78 and/or to actuate the ignition device 38. In some embodiments, the controller 30 may also include a power distributor 32 to distribute power throughout the system, for example, to the solenoid valves at injection ports 48, 58, 68, 78 and to the ignition device 38.

In one or more embodiments, the system 100 includes a user interface 34. The user interface 34 may display any one or more of the measurements from the sensors described above.

In some embodiments, the user interface 34 may be configured to allow customization of the combustion conditions, such as flow rates, pressure, and temperature. The user interface 34 may allow for individual control of each parameter of the system 100 and/or may include pre-programmed functions.

In one or more embodiments, the combustion chamber 10 is maintained at about 100° F. or less before combustion, which helps build pressure once carbon-based nanomaterial composition is produced. After combustion, the temperature within the combustion chamber 10 may be around about 120° F. In some embodiments, a pressure within the combustion chamber 10 is maintained at about 5 to 20 psi prior to combustion. In some embodiments, a pressure within the combustion chamber 10 before combustion is about one half that of a pressure after combustion, for example to about 10 to 40 psi, to facilitate efficient conversion of the carbon-based flue gas into carbon-based nanomaterial composition production.

In some embodiments, the system 100 may be automated to achieve a cost-efficient carbon-based nanomaterial composition production method on- or off-site. The automated system 100 determines the mixture for each internal combustion in the chamber to produce carbon-based nanomaterial composition in real time. In other embodiments, through the use of the user interface 34, the system 100 may be manually controlled.

In any embodiment, the system 100 may be configured to measure, in real-time, the make-up of the carbon-based gas or liquid. Such a measurement may be, for example, derived from the measured temperature and pressure changes within the combustion chamber 10 during and after combustion. The ratios of the carbon-based gas or liquid, hydrogen, and oxygen may be precisely adjusted to achieve a consistent carbon-based nanomaterial composition product, to modify the conversion of carbon from the carbon-based feedstock into carbon-based nanomaterial composition to increase the yield thereof, or ideally, both. After each combustion, the system 100 makes small adjustments as needed to one or more parameters to improve the efficiency of carbon-based nanomaterial composition production. A number of combustions may be required to reach optimal combustion conditions for a given carbon-based gas or liquid. However, the precise control of each of the input reactants allows the system 100 to operate with a wide range of carbon sources-even with a variable carbon source.

Referring now to alternative embodiments, the adipose cell destruction component may further include a secondary targeting composition attached to the carbon-based nanomaterial composition. According to particular embodiments, the secondary targeting composition may include any molecule that is specifically to a desired location within the body. According to certain embodiments, the secondary targeting composition may be coated onto the surface of the carbon-based nanomaterial composition or absorbed in the carbon-based nanomaterial. According to still other embodiments, the secondary targeting composition may act to guide the adipose cell destruction component to the treatment location for destruction of the adipose cells.

Many different aspects and embodiments are possible. Some of those aspects and embodiments are described herein. After reading this specification, skilled artisans will appreciate that those aspects and embodiments are only illustrative and do not limit the scope of the present invention. Embodiments may be in accordance with any one or more of the embodiments as listed below.

Embodiment 1. An adipose cell destruction method comprising: preparing an adipose cell destruction component comprising a carbon-based nanomaterial composition and an adipose cell targeting composition attached to the carbon-based nanomaterial composition; delivering the adipose cell destruction component to a treatment location where the adipose cell targeting composition bonds to adipose cells; and applying a radio frequency to the adipose cell destruction component at the treatment location, wherein the radio frequency is configured to heat the adipose cell destruction component and destroy the adipose cells bonded to the adipose cell targeting composition at the treatment location, wherein the carbon-based nanomaterial composition comprises: a carbon content of at least about 60% and not greater than about 99% based on elemental analysis of the graphene composition, an oxygen content of at least about 1% and not greater than about 35% based on elemental analysis of the graphene composition, and a nitrogen content of at least about 2% and not greater than 50%.

Embodiment 2. An adipose cell destruction component comprising: a carbon-based nanomaterial composition and an adipose cell targeting composition attached to the carbon-based nanomaterial composition; wherein the adipose cell destruction component is configured to be delivered to a treatment location where the adipose cell targeting composition bonds to adipose cells and heated using a radio frequency at the treatment location, wherein heating the adipose cell destruction component destroys the adipose cells bonded to the adipose cell targeting composition at the treatment location, wherein the carbon-based nanomaterial composition comprises: a carbon content of at least about 60% and not greater than about 99% based on elemental analysis of the graphene composition, an oxygen content of at least about 1% and not greater than about 35% based on elemental analysis of the graphene composition, and a nitrogen content of at least about 2% and not greater than 50%.

Embodiment 3. A method of forming an adipose cell destruction component, wherein the method comprises: providing a carbon-based nanomaterial composition, and attaching an adipose cell targeting composition to the carbon-based nanomaterial composition to form the adipose cell destruction component, wherein the adipose cell destruction component is configured to be delivered to a treatment location where the adipose cell targeting composition bonds to adipose cells and heated using a radio frequency at the treatment location, wherein heating the adipose cell destruction component destroys the adipose cells bonded the adipose cell targeting composition, wherein the carbon-based nanomaterial composition comprises: a carbon content of at least about 60% and not greater than about 99% based on elemental analysis of the graphene composition, an oxygen content of at least about 1% and not greater than about 35% based on elemental analysis of the graphene composition, and a nitrogen content of at least about 2% and not greater than 50%.

Embodiment 4. The adipose cell destruction method, adipose cell destruction component, or method of forming an adipose cell destruction component of any one of embodiments 1, 2, and 3, wherein the adipose cell targeting composition comprises a molecule that is specifically attracted to an adipose cell.

Embodiment 5. The adipose cell destruction method, adipose cell destruction component, or method of forming an adipose cell destruction component of any one of embodiments 1, 2, and 3, wherein the treatment location comprises a location within, or on the surface of, a body.

Embodiment 6. The adipose cell destruction method, adipose cell destruction component, or method of forming an adipose cell destruction component of any one of embodiments 1, 2, and 3, wherein the radio frequency applied to the treatment location is at least about 100 MHZ.

Embodiment 7. The adipose cell destruction method, adipose cell destruction component, or method of forming an adipose cell destruction component of any one of embodiments 1, 2, and 3, wherein the carbon-based nanomaterial composition is formed by a method comprising: supplying a gas mixture comprising: acetylene gas at a molar ratio AGₘₒₗ/GMₘₒₗ of at least about 0.20 and not greater than about 0.99, where the AGₘₒₗ is equal to the moles of acetylene gas in the gas mixture and GMₘₒₗ is equal to the total moles of gas in the gas mixture, oxygen gas at a molar ratio OGₘₒₗ/GMₘₒₗ of at least about 0.01 and not greater than about 0.85, where the OGₘₒₗ is equal to the moles of oxygen gas in the gas mixture and GMₘₒₗ is equal to the total moles of gas in the gas mixture, and hydrogen gas at a molar ratio HGₘₒₗ/GMₘₒₗ of at least about 0.05 and not greater than about 0.99, where the HGₘₒₗ is equal to the moles of hydrogen gas in the gas mixture and GMₘₒₗ is equal to the total moles of gas in the gas mixture, igniting the gas mixture to form the carbon-based nanomaterial composition, wherein the carbon-based nanomaterial composition has a carbon hybridization ratio Pₛₚ₃/Pₛₚ₂ of at least about 0.0 and not greater than about 5.0, where Pₛₚ₃ is the percent of carbon within the carbon-based nanomaterial composition having a sp3 hybridization and Pₛₚ₂ is the percent of carbon within the carbon-based nanomaterial composition having a sp2 hybridization.

Embodiment 8. The adipose cell destruction method, adipose cell destruction component, or method of forming an adipose cell destruction component of any one of embodiments 1, 2, and 3, wherein the carbon-based nanomaterial composition comprises: a carbon content of at least about 75% and not greater than about 100% based on elemental analysis of the carbon-based nanomaterial composition, and an oxygen content of at least about 0.0% and not greater than about 25% based on elemental analysis of the carbon-based nanomaterial composition, wherein the carbon-based nanomaterial composition comprises a D/G ratio of at least about 0.1 and not greater than about 2.0; and wherein the carbon-based nanomaterial composition has a carbon hybridization ratio Pₛₚ₃/Pₛₚ₂ of at least about 0.0 and not greater than about 5.0, where Pₛₚ₃ is the percent of carbon within the carbon-based nanomaterial composition having a sp3 hybridization and Pₛₚ₂ is the percent of carbon within the carbon-based nanomaterial composition having a sp2 hybridization.

Embodiment 9. The adipose cell destruction method, adipose cell destruction component, or method of forming an adipose cell destruction component of any one of embodiments 1, 2, and 3, wherein the carbon-based nanomaterial composition comprises: a carbon content of at least about 75% and not greater than about 100% based on elemental analysis of the carbon-based nanomaterial composition, and an oxygen content of at least about 0.0% and not greater than about 25% based on elemental analysis of the carbon-based nanomaterial composition, wherein the carbon-based nanomaterial composition comprises an aspect ratio at least about 1 and not greater than about 50; wherein the carbon-based nanomaterial composition has a carbon hybridization ratio Pₛₚ₃/Pₛₚ₂ of at least about 0.0 and not greater than about 5.0, where Pₛₚ₃ is the percent of carbon within the carbon-based nanomaterial composition having a sp3 hybridization and Pₛₚ₂ is the percent of carbon within the carbon-based nanomaterial composition having a sp2 hybridization.

Embodiment 10. The adipose cell destruction method, adipose cell destruction component, or method of forming an adipose cell destruction component of any one of embodiments 1, 2, and 3, wherein the carbon-based nanomaterial composition comprises a carbon content of at least about 75% based on elemental analysis of the carbon-based nanomaterial composition.

Embodiment 11. The adipose cell destruction method, adipose cell destruction component, or method of forming an adipose cell destruction component of any one of embodiments 1, 2, and 3, wherein the carbon-based nanomaterial composition comprises a carbon content of not greater than about 100% based on elemental analysis of the carbon-based nanomaterial composition.

Embodiment 12. The adipose cell destruction method, adipose cell destruction component, or method of forming an adipose cell destruction component of any one of embodiments 1, 2, and 3, wherein the carbon-based nanomaterial composition comprises an oxygen content of at least about 0.0% based on elemental analysis of the carbon-based nanomaterial composition.

Embodiment 13. The adipose cell destruction method, adipose cell destruction component, or method of forming an adipose cell destruction component of any one of embodiments 1, 2, and 3, wherein the carbon-based nanomaterial composition comprises an oxygen content of not greater than about 25.0% based on elemental analysis of the carbon-based nanomaterial composition.

Embodiment 14. The adipose cell destruction method, adipose cell destruction component, or method of forming an adipose cell destruction component of any one of embodiments 1, 2, and 3, wherein the carbon-based nanomaterial composition comprises a carbon hybridization ratio Pₛₚ₃/Pₛₚ₂ of at least about 0.0, where Pₛₚ₃ is the percent of carbon within the carbon-based nanomaterial composition having a sp3 hybridization and Pₛₚ₂ is the percent of carbon within the carbon-based nanomaterial composition having a sp2 hybridization.

Embodiment 15. The adipose cell destruction method, adipose cell destruction component, or method of forming an adipose cell destruction component of any one of embodiments 1, 2, and 3, wherein the carbon-based nanomaterial composition has a carbon hybridization ratio Pₛₚ₃/Pₛₚ₂ of not greater than about 5.0, where Pₛₚ₃ is the percent of carbon within the carbon-based nanomaterial composition having a sp3 hybridization and Pₛₚ₂ is the percent of carbon within the carbon-based nanomaterial composition having a sp2 hybridization.

Embodiment 16. The adipose cell destruction method, adipose cell destruction component, or method of forming an adipose cell destruction component of any one of embodiments 1, 2, and 3, wherein the carbon-based nanomaterial composition comprises a D/G ratio of not greater than about 0.1.

Embodiment 17. The adipose cell destruction method, adipose cell destruction component, or method of forming an adipose cell destruction component of any one of embodiments 1, 2, and 3, wherein the carbon-based nanomaterial composition comprises a D/G ratio of at least about 2.0.

Embodiment 18. The adipose cell destruction method, adipose cell destruction component, or method of forming an adipose cell destruction component of any one of embodiments 1, 2, and 3, wherein the carbon-based nanomaterial composition comprises an aspect ratio of not greater than about 50.

Embodiment 19. The adipose cell destruction method, adipose cell destruction component, or method of forming an adipose cell destruction component of any one of embodiments 1, 2, and 3, wherein the carbon-based nanomaterial composition comprises an aspect ratio of at least about 1.

Embodiment 20. The adipose cell destruction method, adipose cell destruction component, or method of forming an adipose cell destruction component of any one of embodiments 1, 2, and 3, wherein the carbon-based nanomaterial composition is formed from a gas mixture.

Embodiment 21. The adipose cell destruction method, adipose cell destruction component, or method of forming an adipose cell destruction component of any one of embodiments 1, 2, and 3, wherein the gas mixture comprises acetylene gas at a concentration of at least about 1.2 mol.

Embodiment 22. The adipose cell destruction method, adipose cell destruction component, or method of forming an adipose cell destruction component of any one of embodiments 1, 2, and 3, wherein the gas mixture comprises acetylene gas at a concentration of not greater than about 18 mol.

Embodiment 23. The adipose cell destruction method, adipose cell destruction component, or method of forming an adipose cell destruction component of any one of embodiments 1, 2, and 3, wherein the gas mixture comprises oxygen gas at a concentration of at least about 0.3 mol.

Embodiment 24. The adipose cell destruction method, adipose cell destruction component, or method of forming an adipose cell destruction component of any one of embodiments 1, 2, and 3, wherein the gas mixture comprises oxygen gas at a concentration of not greater than about 12 mol.

Embodiment 25. The adipose cell destruction method, adipose cell destruction component, or method of forming an adipose cell destruction component of any one of embodiments 1, 2, and 3, wherein the gas mixture comprises hydrogen gas at a concentration of at least about 0.6 mol.

Embodiment 26. The adipose cell destruction method, adipose cell destruction component, or method of forming an adipose cell destruction component of any one of embodiments 1, 2, and 3, wherein the gas mixture comprises hydrogen gas at a concentration of not greater than about 20.0 mol.

Embodiment 27. The adipose cell destruction method, adipose cell destruction component, or method of forming an adipose cell destruction component of any one of embodiments 1, 2, and 3, wherein the gas mixture comprises methane gas at a molar ratio MGₘₒₗ/GMₘₒₗ of at least about 0.25 and not greater than about 0.99, where the MGₘₒₗ is equal to the moles of methane gas in the gas mixture and GMₘₒₗ is equal to the total moles of gas in the gas mixture.

Embodiment 28. The adipose cell destruction method, adipose cell destruction component, or method of forming an adipose cell destruction component of any one of embodiments 1, 2, and 3, wherein the gas mixture comprises methane gas at a concentration of at least about 1.2 mol.

Embodiment 29. The adipose cell destruction method, adipose cell destruction component, or method of forming an adipose cell destruction component of any one of embodiments 1, 2, and 3, wherein the gas mixture comprises methane gas at a concentration of not greater than about 18 mol.

Embodiment 30. The adipose cell destruction method, adipose cell destruction component, or method of forming an adipose cell destruction component of any one of embodiments 1, 2, and 3, wherein the carbon-based nanomaterial composition is formed in a system for carbon-based nanomaterial composition synthesis, wherein the system comprises: an enclosed chamber comprising a hollow interior; a carbon-based gas source fluidically coupled to the chamber and configured to supply a carbon-based gas to the hollow interior; a hydrogen source that is independent of the carbon-based gas source and that is fluidically coupled to the chamber and configured to supply hydrogen to the hollow interior; an oxygen source that is independent of the carbon-based gas source and that is fluidically coupled to the chamber and configured to supply oxygen to the hollow interior; an igniter configured to ignite the carbon-based gas, hydrogen, and oxygen in the hollow interior; a first flow meter coupled to the carbon-based gas source, a second flow meter coupled to the hydrogen source, a third flow meter coupled to the oxygen source; and a controller in communication with and configured to receive flow data from the first, second, and third flow meters; wherein the controller is configured to adjust flow from one or more of the carbon-based gas source, the hydrogen source, and/or the oxygen source in response to the flow data.

Embodiment 31. The adipose cell destruction method, adipose cell destruction component, or method of forming an adipose cell destruction component of embodiment 30, wherein the carbon-based gas is a flue gas resulting from an industrial reaction process.

Embodiment 32. The adipose cell destruction method, adipose cell destruction component, or method of forming an adipose cell destruction component of embodiment 31, wherein the industrial reaction process is a coal energy plant, a drilling operation, a combustion engine, or a landfill.

Embodiment 33. The adipose cell destruction method, adipose cell destruction component, or method of forming an adipose cell destruction component of embodiment 31, wherein the carbon-based gas source comprises a storage tank, an inlet line, and an outlet line; wherein the storage tank is coupled to the chamber via the outlet line; and wherein the flue gas is directed from the industrial reaction process through the inlet line to the storage tank.

Embodiment 34. The adipose cell destruction method, adipose cell destruction component, or method of forming an adipose cell destruction component of embodiment 31, wherein the chamber is co-located with the industrial reaction process.

Embodiment 35. The adipose cell destruction method, adipose cell destruction component, or method of forming an adipose cell destruction component of embodiment 30, further comprising an inert gas source fluidically coupled to the chamber and configured to supply an inert gas to the hollow interior.

Embodiment 36. The adipose cell destruction method, adipose cell destruction component, or method of forming an adipose cell destruction component of embodiment 30, wherein the carbon-based gas source is coupled to the chamber via a first one-way valve, the hydrogen source is coupled to the chamber via a second one-way valve, and the oxygen source is coupled to the chamber via a third one-way valve.

Embodiment 37. The adipose cell destruction method, adipose cell destruction component, or method of forming an adipose cell destruction component of embodiment 36, wherein the chamber further comprises an exhaust valve.

Embodiment 38. The adipose cell destruction method, adipose cell destruction component, or method of forming an adipose cell destruction component of embodiment 30, further comprising a pressure sensor configured to measure a pressure within the hollow interior and a temperature sensor configured to measure a temperature within the hollow interior; wherein the controller is in communication with and configured to receive pressure data from the pressure sensor; wherein the controller is in communication with and configured to receive temperature data from the temperature sensor; and wherein the controller is configured to adjust flow from one or more of the carbon-based gas source, the hydrogen source, and the oxygen source in response to the flow data, the pressure data, the temperature data, or a combination thereof.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Separate embodiments may also be provided in combination in a single embodiment, and conversely, various features that are, for brevity, described in the context of a single embodiment, may also be provided separately or in any subcombination. Further, reference to values stated in ranges includes each and every value within that range. Many other embodiments may be apparent to skilled artisans only after reading this specification. Other embodiments may be used and derived from the disclosure, such that a structural substitution, logical substitution, or another change may be made without departing from the scope of the disclosure. Accordingly, the disclosure is to be regarded as illustrative rather than restrictive.

What is claimed is:

1. An adipose cell destruction method comprising:
preparing an adipose cell destruction component comprising a carbon-based nanomaterial composition and an adipose cell targeting composition attached to the carbon-based nanomaterial composition;
delivering the adipose cell destruction component to a treatment location where the adipose cell targeting composition bonds to adipose cells; and
applying a radio frequency to the adipose cell destruction component at the treatment location, wherein the radio frequency is configured to heat the adipose cell destruction component and destroy the adipose cells bonded to the adipose cell targeting composition at the treatment location,
wherein the carbon-based nanomaterial composition comprises:
a carbon content of at least about 60% and not greater than about 99% based on elemental analysis of the graphene composition,
an oxygen content of at least about 1% and not greater than about 35% based on elemental analysis of the graphene composition, and
a nitrogen content of at least about 2% and not greater than 50%.

2. The adipose cell destruction method of claim 1, wherein the adipose cell targeting composition comprises a molecule that is specifically attracted to an adipose cell.

3. The adipose cell destruction method of claim 1, wherein the treatment location comprises a location within, or on the surface of, a body.

4. The adipose cell destruction method of claim 1, wherein the radio frequency applied to the treatment location is at least about 100 MHz.

5. The adipose cell destruction method of claim 1, wherein the carbon-based nanomaterial composition is formed by a method comprising:
supplying a gas mixture comprising:
acetylene gas at a molar ratio AGₘₒₗ/GMₘₒₗ of at least about 0.20 and not greater than about 0.99, where the AGₘₒₗ is equal to the moles of acetylene gas in the gas mixture and GMₘₒₗ is equal to the total moles of gas in the gas mixture,
oxygen gas at a molar ratio OGₘₒₗ/GMₘₒₗ of at least about 0.01 and not greater than about 0.85, where the OGₘₒₗ is equal to the moles of oxygen gas in the gas mixture and GMₘₒₗ is equal to the total moles of gas in the gas mixture, and
hydrogen gas at a molar ratio HGₘₒₗ/GMₘₒₗ of at least about 0.05 and not greater than about 0.99, where the HGₘₒₗ is equal to the moles of hydrogen gas in
the gas mixture and GMₘₒₗ is equal to the total moles of gas in the gas mixture, igniting the gas mixture to form the carbon-based nanomaterial composition,
wherein the carbon-based nanomaterial composition has a carbon hybridization ratio Pₛₚ₃/Pₛₚ₂ of at least about 0.0 and not greater than about 5.0, where Pₛₚ₃ is the percent of carbon within the carbon-based nanomaterial composition having a sp3 hybridization and Pₛₚ₂ is the percent of carbon within the carbon-based nanomaterial composition having a sp2 hybridization.

6. The adipose cell destruction method of claim 1, wherein the carbon-based nanomaterial composition comprises:
a carbon content of at least about 75% and not greater than about 100% based on elemental analysis of the carbon-based nanomaterial composition, and
an oxygen content of at least about 0.0% and not greater than about 25% based on elemental analysis of the carbon-based nanomaterial composition,
wherein the carbon-based nanomaterial composition comprises a D/G ratio of at least about 0.1 and not greater than about 2.0; and
wherein the carbon-based nanomaterial composition has a carbon hybridization ratio Pₛₚ₃/Pₛₚ₂ of at least about 0.0 and not greater than about 5.0, where Pₛₚ₃ is the percent of carbon within the carbon-based nanomaterial composition having a sp3 hybridization and Pₛₚ₂ is the percent of carbon within the carbon-based nanomaterial composition having a sp2 hybridization.

7. The adipose cell destruction method of claim 1, wherein the carbon-based nanomaterial composition comprises:
a carbon content of at least about 75% and not greater than about 100% based on elemental analysis of the carbon-based nanomaterial composition, and
an oxygen content of at least about 0.0% and not greater than about 25% based on elemental analysis of the carbon-based nanomaterial composition,
wherein the carbon-based nanomaterial composition comprises an aspect ratio at least about 1 and not greater than about 50;
wherein the carbon-based nanomaterial composition has a carbon hybridization ratio Pₛₚ₃/Pₛₚ₂ of at least about 0.0 and not greater than about 5.0, where Pₛₚ₃ is the percent of carbon within the carbon-based nanomaterial composition having a sp3 hybridization and Pₛₚ₂ is the percent of carbon within the carbon-based nanomaterial composition having a sp2 hybridization.

8. The adipose cell destruction method of claim 1, wherein the carbon-based nanomaterial composition comprises a carbon content of at least about 75% based on elemental analysis of the carbon-based nanomaterial composition.

9. The adipose cell destruction method of claim 1, wherein the carbon-based nanomaterial composition comprises a carbon content of not greater than about 100% based on elemental analysis of the carbon-based nanomaterial composition.

10. The adipose cell destruction method of claim 1, wherein the carbon-based nanomaterial composition comprises an oxygen content of at least about 0.0% based on elemental analysis of the carbon-based nanomaterial composition.

11. The adipose cell destruction method of claim 1, wherein the carbon-based nanomaterial composition comprises an oxygen content of not greater than about 25.0% based on elemental analysis of the carbon-based nanomaterial composition.

12. The adipose cell destruction method of claim 1, wherein the carbon-based nanomaterial composition comprises a carbon hybridization ratio Pₛₚ₃/Pₛₚ₂ of at least about 0 and not greater than about 5.0, where Pₛₚ₃ is the percent of carbon within the carbon-based nanomaterial composition having a sp3 hybridization and Pₛₚ₂ is the percent of carbon within the carbon-based nanomaterial composition having a sp2 hybridization.

13. The adipose cell destruction method of claim 1, wherein the carbon-based nanomaterial composition comprises a D/G ratio of not greater than about 0.1 and not greater than about 2.0.

14. An adipose cell destruction component comprising:
a carbon-based nanomaterial composition and an adipose cell targeting composition attached to the carbon-based nanomaterial composition;
wherein the adipose cell destruction component is configured to be delivered to a treatment location where the adipose cell targeting composition bonds to adipose cells and heated using a radio frequency at the treatment location,
wherein heating the adipose cell destruction component destroys the adipose cells bonded to the adipose cell targeting composition at the treatment location,
wherein the carbon-based nanomaterial composition comprises:
a carbon content of at least about 60% and not greater than about 99% based on elemental analysis of the graphene composition,
an oxygen content of at least about 1% and not greater than about 35% based on elemental analysis of the graphene composition, and
a nitrogen content of at least about 2% and not greater than 50%.

15. The adipose cell destruction component of claim 14, wherein the adipose cell targeting composition comprises a molecule that is specifically attracted to an adipose cell.

16. The adipose cell destruction component of claim 14, wherein the treatment location comprises a location within, or on the surface of, a body.

17. The adipose cell destruction component of claim 14, wherein the radio frequency applied to the treatment location is at least about 100 MHz.

18. The adipose cell destruction component of claim 14, wherein the carbon-based nanomaterial composition is formed by a method comprising:
supplying a gas mixture comprising:
acetylene gas at a molar ratio AGₘₒₗ/GMₘₒₗ of at least about 0.20 and not greater than about 0.99, where the AGₘₒₗ is equal to the moles of acetylene gas in the gas mixture and GMₘₒₗ is equal to the total moles of gas in the gas mixture,
oxygen gas at a molar ratio OGₘₒₗ/GMₘₒₗ of at least about 0.01 and not greater than about 0.85, where the OGₘₒₗ is equal to the moles of oxygen gas in the gas mixture and GMₘₒₗ is equal to the total moles of gas in the gas mixture, and
hydrogen gas at a molar ratio HGₘₒₗ/GMₘₒₗ of at least about 0.05 and not greater than about 0.99, where the HGₘₒₗ is equal to the moles of hydrogen gas in
the gas mixture and GMₘₒₗ is equal to the total moles of gas in the gas mixture, igniting the gas mixture to form the carbon-based nanomaterial composition,
wherein the carbon-based nanomaterial composition has a carbon hybridization ratio Pₛₚ₃/Pₛₚ₂ of at least about 0.0 and not greater than about 5.0, where Pₛₚ₃ is the percent of carbon within the carbon-based nanomaterial composition having a sp3 hybridization and Pₛₚ₂ is the percent of carbon within the carbon-based nanomaterial composition having a sp2 hybridization.

19. The adipose cell destruction component of claim 14, wherein the carbon-based nanomaterial composition comprises:
a carbon content of at least about 75% and not greater than about 100% based on elemental analysis of the carbon-based nanomaterial composition, and
an oxygen content of at least about 0.0% and not greater than about 25% based on elemental analysis of the carbon-based nanomaterial composition,
wherein the carbon-based nanomaterial composition comprises a D/G ratio of at least about 0.1 and not greater than about 2.0; and
wherein the carbon-based nanomaterial composition has a carbon hybridization ratio Pₛₚ₃/Pₛₚ₂ of at least about 0.0 and not greater than about 5.0, where Pₛₚ₃ is the percent of carbon within the carbon-based nanomaterial composition having a sp3 hybridization and Pₛₚ₂ is the percent of carbon within the carbon-based nanomaterial composition having a sp2 hybridization.

20. A method of forming an adipose cell destruction component, wherein the method comprises:
providing a carbon-based nanomaterial composition, and
attaching an adipose cell targeting composition to the carbon-based nanomaterial composition to form the adipose cell destruction component,
wherein the adipose cell destruction component is configured to be delivered to a treatment location where the adipose cell targeting composition bonds to adipose cells and heated using a radio frequency at the treatment location,
wherein heating the adipose cell destruction component destroys the adipose cells bonded the adipose cell targeting composition,
wherein the carbon-based nanomaterial composition comprises:
a carbon content of at least about 60% and not greater than about 99% based on elemental analysis of the graphene composition,
an oxygen content of at least about 1% and not greater than about 35% based on elemental analysis of the graphene composition, and
a nitrogen content of at least about 2% and not greater than 50%.
